(19) United States
(12) Patent Application Publication
(10) Pub. No.: US 2001/0007758 A1
(43) Pub. Date: Jul. 12, 2001

(54) Treatment of multiple sclerosis using COP-1 and Th2-enhancing cytokines

(75) Inventors: Howard L. Weiner, Brookline, MA (US); Ruth Marron, Brookline, MA (US); Anthony Slavin, Boston, MA (US)

Correspondence Address: DARBY & DARBY P.C., 805 Third Avenue, New York, NY, 10022, US

(73) Assignee: Autoimmune, Inc.

(21) Appl. No.: 09/777,582

(22) Filed: Feb. 6, 2001

Related U.S. Application Data
(63) Continuation of application No. 09/250,360, filed on Feb. 12, 1999.
(60) Provisional application No. 60/074,696, filed on Feb. 13, 1998.

(51) Int. Cl. A61K 38/00; C12P 21/06; A61K 45/00

(52) U.S. Cl. 435/69.1; 424/85.2; 514/12

(57) ABSTRACT

The invention relates to a treatment for multiple sclerosis. COP-1 (copolymer-1), a synthetic polymer consisting of a mixture of random synthetic polypeptides composed of L-alanine, L-glutamic acid, L-lysine and L-tyrosine in a molar ratio of about 6:2:5:1, is administered mucosally to patients afflicted with the disease in combination with Th2 enhancing cytokines such as IL-4 or IL-10. The combination treatment of IL-4 or IL-10 (preferably orally administered) with mucosally administered COP-1 shows a substantially greater suppressive effect than does treatment with cytokine or COP-1 alone.

### CROSS-REFERENCE TO RELATED APPLICATIONS

This application claims priority pursuant to 35 U.S.C. 119 based upon Provisional Application Ser. No. 60/074,696 filed Feb. 13, 1998, the entire disclosure of which is hereby incorporated by reference.

### FIELD OF THE INVENTION

This invention pertains to an improvement in the ability to reduce autoimmune reactions associated with Multiple Sclerosis.

### BACKGROUND OF THE INVENTION

Autoimmune diseases are characterized by an abnormal immune response directed to self or autologous tissues. Based on the type of immune response (or immune reaction) involved, autoimmune diseases in mammals can generally be classified into one of two different types: cell-mediated (i.e., T-cell-mediated) or antibody-mediated disorders. Multiple sclerosis (MS) is a T-cell mediated autoimmune disease. (Trapp et al. New Eng. J. Med. 338(5):278 (1998)). More than 1,000,000 young adults worldwide between the ages of thirty and forty have MS. MS is the most common disease of the central nervous system and is the most common cause of neurological disability in young adults. Pathophysiologically, circulating autoreactive T cells mediate much of the central nervous system destruction seen in MS patients. (Rudick et al. New Eng. J. Med. 337:1604(1997)).

In MS, T-cells react with myelin basic protein (MBP) which is a component of myelin in the central nervous system. The demonstration that activated T-cells specific for MBP can be isolated from MS patients supports the proposition that MS is an autoimmune disease wherein T-cells destroy the self or autologous neural tissue (Allegretta et al. Science: 247: 778 (1990)).

Experimental allergic encephalomyelitis (EAE) is the primary animal model for MS. EAE can readily be induced in small mammals by immunization with MBP in an appropriate adjuvant or by passive transfer of CD4+, MBP-reactive T-cells (Alvord Jr, E. C., et al. eds. in Experimental Allergic Encephalomyelitis a Useful Model for Multiple Sclerosis, A. R. Liss, N.Y., 1984; Makhtarian et al. Nature 309: 356 (1984); Ben-Nun et al. J. Immunol. 129:303 (1982)). The T-cells that induce EAE in both mice and rats recognize peptides corresponding to immunodominant regions of MBP presented by antigen-presenting cells on class II Major Histocompatibility Complex (MHC) molecules.

MS is currently treated with a certain anti-inflammatory and immunosuppressive agents, such agents include: (i) corticosteroids, which have both immunomodulatory and immunosuppressive effects; (ii) interferon-β; (iii) glatiramer acetate (COP-1); (iv) azathioprine, a purine analog which depresses both cell-mediated and humoral immunity; (v) intravenous immune globulin; (vi) methotrexate, which inhibits dihydrofolate reductase and depresses cell-mediated and humoral immunity; (vii) cyclophosphamide, an alkylating agent which has cytotoxic and immunosuppressive effects; and, (viii) cyclosporine, which has potent immunosuppressive effects by inhibiting T cell activation. Despite treatment with such anti-inflammatory or immunosuppressive drugs, more than 50% of the patients with MS steadily deteriorate as a result of focal destruction of the spinal cord, cerebellum, and cerebral cortex.

Many of the currently used drugs have limited long-term efficacy, in part, because they have significant cytotoxic effects. For example, prolonged treatment with cyclophosphamide can lead to alopecia, nausea, vomiting, hemorrhagic cystitis, leukopenia, myocarditis, infertility, and pulmonary interstitial fibrosis. Treatment with immunosuppressive agents can eventually induce “global” immunosuppression in the treated patient, which greatly increase the risk of infection. Patients subjected to prolonged global immunosuppression have an increased risk of developing severe medical complications from treatment, such as malignancies, kidney failure and diabetes.

An alternative approach to the treatment of MS is the use of intravenous or oral administration of MBP to modulate T-cell immune response. Intravenous administration of MBP or fragments thereof containing immunodominant epitopes of MBP suppresses the immune system by causing clonal anergy, or T-cell unresponsiveness, which deactivates T-cells specific for MBP. The end-result is that MBP-specific T cells no longer proliferate in response to MBP. The inability of the T-cell to proliferate results in a decrease in T-cell mediated destruction of neural tissues. Oral administration of autoantigens such as MBP suppresses immune response against MBP via active suppression or anergy, depending upon the dose administered. Oral administration of MBP in a single dose and in substantially larger amounts than those that trigger active suppression, can also induce tolerance through clonal deletion.

An immunochemical analog of MBP that is effective in treating MS is glatiramer acetate, or copolymer-1 (COP-1) (U.S. Pat. No. 3,849,550; PCT Application WO/95/31990). COP-1, in its commercially available form, is a mixture of random synthetic polypeptides composed of L-alanine, L-glutamic acid, L-lysine and L-tyrosine in a molar ratio of 6.0:1.9:4.7:1.0. It was first synthesized as an immunochemical mimic of MBP. For example, certain monoclonal antibodies to COP-1 cross-react with MBP (Teitelbaum et al. Proc. Natl. Acad. Sci. USA 88:9258 (1991)). Also, COP-1 has been found to induce T suppressor cells specific for MBP (Lando et al. J. Immunol. 123:2156 (1979)). Experiments in mice indicate that COP-1 also specifically inhibits MBP-specific T cells that are involved in the destruction of central nervous system tissue in EAE (Teitelbaum et al. Proc. Natl. Acad. USA 85:9724 (1995)).

Although COP-1 is immunologically similar to MBP the linear amino acid sequence for COP-1 has no known homology with the amino acid sequence of MBP. Furthermore, COP-1 is immunologically different from MBP in certain ways. For example, COP-1 is not encephalitogenic, i.e., it does not cause experimental allergic encephalitis (EAE) when injected, whereas MBP is highly encephalitogenic (Teitelbaum et al. Eur. J. Immunol. 4:242 (1971)). Also, lack of immunological cross-reactivity was observed by Burns et al. Neurology 36:92 (1986).

Administration of COP-1 may: (i) increase the percentage of NK cells; (ii) reduce serum IL-2 receptors; (iii) suppress TNF-α; and, (iv) increase TGF-β and IL-4 (Ariel et al. Multiple Sclerosis 3(5), S053 (1997)).

Patients with MS have been successfully treated with parenterally administered COP-1 (Bornstein et al. Transactions American Neurological Association, 348 (1987)). Patients were injected daily with subcutaneous injections of COP-1 of 20 mg (Bornstein et al. Annals of Neurology 11:17 (1981)). In the treated patients, (i) the annualized relapse rate was 29% lower, (ii) the proportion of patients that did not have a relapse in clinical disease was higher (34 percent vs. 27 percent), and (iii) the treated group had a significant improvement on their Expanded Disability Status Scale—a standard clinical measure of physical function in MS patients.

Recent studies indicate that COP-1 is effective for treating EAE when administered orally (Teitelbaum et al. Multiple Sclerosis 3(5), P169 (1997)). Oral administration of COP-1 to rats, (i) suppressed the severity and incidence of EAE, (ii) inhibited T cell proliferative responses, and (iii) inhibited TH1 cytokine production.

Autoimmune disease can be treated by oral administration of bystander antigens. Such treatment proceeds through an active suppression mechanism. This method is discussed extensively in PCT Application PCT/US93/01705 (published as WO 93/16724) and involves the oral administration of antigens specific for the tissue under autoimmune attack.

Oral administration of bystander antigens elicits regulatory (suppressor) T-cells (which can be of the CD4+or CD8+ type) that are targeted to the organ or tissue under attack, where they cause the release of at least one antigen-nonspecific immunosuppressive factor or immunoregulatory cytokine (such as TGF-β, IL-4 or IL-10), thereby suppressing the local immune response.

Specifically, oral treatment with “bystander antigens” causes regulatory (suppressor) T-cells to be induced in the gut-associated lymphoid tissue (GALT), or, in the case of by-inhalation administration, mucosa associated lymphoid tissue (MALT). These regulatory cells are released in the blood or lymphatic tissue and then migrate to the organ or tissue affected by the autoimmune disease. There the T-cells can suppress autoimmune attack of the affected organ or tissue. T-cells elicited by the bystander antigens are targeted to the locus of autoimmune attack where they mediate the local release of certain immunomodulatory factors and cytokines, such as transforming growth factor beta (TGF-β) interleukin-4 (IL-4) or interleukin-10 (IL-10). Of these, TGF-β is an antigen-nonspecific immunosuppressive factor in that it suppresses all immune attack regardless of the antigen that triggers its release. Because oral tolerization with bystander antigen can cause release of TGF-β only in the vicinity of autoimmune attack, there is no systemic immunosuppression. IL-4 and IL-10 are also antigen-nonspecific immunoregulatory cytokines. That is, IL-4 in particular enhgances Th2 response by acting on T-cell precursors. This causes the T-cells to differentiate preferentially into Th2 cells. Th2 cells produce a wide range of cytokines, including, but not limited to IL-4, IL-5, IL-6, and IL-10. These cytokines regulate production of various immunoglobulin classes, e.g., IgG1, by B lymphocytes. Th2 cells can also diminish the potency of the cellular immune response initiated by other effector arms of the immune system (Paul, W. E., Fundamental Immunology, Raven Press, pg 13-14, 1993).

Administration of Th2-enhancing cytokines in combination with MBP augments the suppressive effect of MBP in terms of both disease incidence and the delay of the onset in EAE (PCT/US95/04512, published as WO 95/27500). For example, EAE was induced in SJL/J mice by immunizing with 0.4 mg of mouse MBP, together with Mycobacterium tuberculosis and pertussis toxin at the appropriate intervals. The mice were divided into several experimental groups which were fed orally the following agents: (i) hen egg lysozyme (HEL) as a control; (ii) mouse IL-4; (iii) mouse MBP; or, (iv) MBP plus IL-4. Animals were monitored for disease onset for 35 days. Treatment with a combination of oral IL-4 (1000 units) and MBP reduced both disease onset and clinical score. It also delayed the onset of disease. In fact, the delay in disease onset was substantially greater (30 days) with the combination treatment than with either IL-4 or MBP alone (21 and 22 days, respectively).

To date there has been no teaching known to the inventors that oral COP-1 can be combined with administration of IL-4 or IL-10 to obtain an effective treatment of MS. Nor is it known whether combining administration of a Th2 cytokine with oral administration of other autoimmune suppressive agents in general is of benefit in treating EAE or MS. While COP-1 shares certain immunological properties with MBP, it has a random amino acid sequence and is not known to have any structural similarity to MBP. Furthermore, it COP-1 differs from MBP in certain of its immunological properties. It therefore was not predictable whether the combination of mucosally administered COP-1 with mucosal or parenterally administered IL-4 or IL-10 would be effective in the treatment of MS or EAE.

Accordingly, one object of the present invention is to provide an improved and/or more convenient method for treating mammals suffering from MS.

An additional object of the present invention is to provide an improved method for treating mammals suffering from MS that can, if desired, be administered exclusively via the oral route.

A third object of the invention is to provide a method for treating mammals suffering from MS that provides an adjunct therapy for COP-1 administration.

### SUMMARY OF THE INVENTION

It has now been found that a combination of (i) mucosal administration of COP-1 and (ii) administration of a polypeptide having Th2-enhancing cytokine activity is substantially more effective than the administration of COP-1 alone, or of the peptide having Th2-enhancing cytokine activity alone in suppressing autoimmune reaction associated with MS. It has been determined in particular that mucosal or parenteral administration of IL-4 or IL-10 combined with mucosal administration of COP-1 is of benefit in the treatment of MS.

### DETAILED DESCRIPTION OF THE INVENTION

All patent applications, patents, and literature references cited in this specification are hereby incorporated by reference in their entirety. In case of any conflict, the definitions and interpretations of the present disclosure are intended to prevail.

### Definitions

The following terms, when used in this disclosure, are intended to have the meanings ascribed to them below:

“Th2-enhancing cytokines” are naturally occurring antigen-nonspecific immunoregulatory substances that: (i) are normally secreted or induced by regulatory immune system cells; and, (ii) enhance the frequency of Th2 cells (and/or inhibit Th1 cells).

“Mammal” is defined herein as any warm-blooded organism which gives birth to live babies, having an immune system and being susceptible to an autoimmune disease.

“Treatment” is intended to include both treatment to prevent or delay the onset of any manifestation, clinical or subclinical, e.g., histological, symptoms thereof of Multiple Sclerosis, as well as the therapeutic suppression or alleviation of symptoms after their manifestation by abating autoimmune attack and preventing or slowing down autoimmune tissue destruction. “Abatement”, “suppression” or “reduction” of autoimmune attack or reaction encompasses partial reduction or amelioration of one or more symptoms of the attack or reaction. A “substantially” increased suppressive effect (or abatement or reduction) of the “autoimmune reaction” means a significant decrease in one or more markers or histological or clinical indicators of MS. Non-limiting examples are a reduction by at least 1 unit in limb paralysis score.

As used in the present specification, administration of a Th2-enhancing cytokine “in conjunction with”, or “in association with”, or “combined with” administration of COP-1 means before, substantially simultaneously with, or after administration of COP-1. “Substantially simultaneously” means within the same 24-hour period, and preferably within one hour before or after.

“Oral” administration includes oral, enteral or intragastric administration.

“Mucosal” administration includes oral, enteral, intragastric, intra-nasal, by-inhalation, and buccal administration, and any other form of administration that results in exposure of mucosal associated lymphoid tissue (MALT) to antigens. Administration to gastrointestinal associated lymphoid tissue (GALT) is intended to be included within “mucosal administration”.

“Parenteral” administration includes subcutaneous, intradermal, intramuscular, intravenous, intraperitoneal or intrathecal administration.

### Animal Models

Throughout the present specification, reference is made to a model system that has been developed for studying MS: EAE. Those of ordinary skill in the art recognize that many of the potential immune therapies for MS are first tested in this animal model system. The disease is induced by immunization with MBP or proteolipid protein (PLP) and an adjuvant (such as Freund's Complete Adjuvant, “CFA”). The antigen that is used to induce the disease is the autoantigen, MBP or PLP. Immunization with either antigen induces either a monophasic or an exacerbating/remitting form of demyelinating disease (depending on the type and species of rodent and well-known details of induction). The induced disease has many of the characteristics of the autoimmune disease components of MS and therefore serves as an animal model for the disease. Furthermore, the successful treatment of EAE by oral tolerization, and the parallel success in decreasing the frequency of disease-inducing cells in humans, and, in many cases, ameliorating the symptoms of MS, using oral administration of myelin, has validated the use of EAE as a model system for predicting the success of different oral tolerization regimens.

The above disclosed model system is employed to demonstrate the efficacy and improved treatment provided by the present invention. The model is particularly suitable for testing therapies because the immunological mechanisms in EAE are closely parallel to those in MS. In the case of oral tolerization, the suppression of autoimmunity obtained in the model is independent of actual or potential differences between human MS autoimmune disorder and the animal model. The model is particularly suitable for testing therapies based on use of Th2-enhancing cytokines because such cytokines generally have the same or similar activities in animal models as in humans.

### Preparation of COP-1, IL-4 and IL-10

According to the present invention, mucosal administration of COP-1 together with mucosal or parenteral administration of a peptide having Th-2 enhancing cytokine activity is used to suppress autoimmune reaction associated with MS.

COP-1, according to the present invention, may be prepared by methods known in the art. For example, COP-1 may be prepared by the process disclosed in U.S. Pat. No. 3,849,550, wherein the N-carboxyanhydrides of tyrosine, alanine, γ-benzyl glutamate and ε-N-trifluoro-acetyllysine are polymerized at ambient temperature in anhydrous dioxane with diethylamine as an inhibitor. The deblocking of the γ-carboxyl group of the glutamic acids is carried out with hydrogen bromide in glacial acetic acid and is followed by the removal of the trifluoracetyl groups from the lysine residues by 1M piperidine. The resulting mixture of polypeptides consists essentially of polymers of alanine, glutamic acid, lysine, and tyrosine, in a molar ratio of about 6:2:5:1.

COP-1 is also available commercially from Teva Pharmaceuticals, Kfar-Saba, Israel.

COP-1 may be prepared for use in the invention in any of the forms which maintain its therapeutic utility. These include mixtures of peptides having various molecular weight ranges. COP-1 having a desired molecular weight range can be obtained by methods known in the art. Such methods include gel filtration high pressure liquid chromatography of COP-1 to remove high molecular weight species as disclosed in WO 95/31990. In one embodiment, the COP-1 has about 75% of its polymer species within the molecular weight range of about 2 KDa to about 20 KDa. In another embodiment, COP-1 has an average molecular weight from about 4 KDa to 9 KDa. It is understood that COP-1 may be subjected to enzymatic or other degradation in order to comprise polymer species of a length different from, or otherwise modified, from conventional COP-1 according to the known methods.

In the preferred embodiment, COP-1 is administered in combination with IL-4 or IL-10. IL-4 and IL-10 are commercially available from Pharmingen, San Diego, Calif. They can also be isolated from natural sources (T cells) that normally produce either cytokine (John E. Coligan et al. eds., Current Protocols in Immunology, Volume 1, Chapter 6, John H. Wiley & Sons, Inc., 1997). Both cytokines can also be obtained using recombinant DNA technology, in bacterial, yeast, insect and mammalian cells, using techniques well-known to those of ordinary skill in the art. For example, the DNA sequence encoding human IL-4 is disclosed in Yokota et al., Proc. Natl. Acad. Sci. USA 83:5894 (1986).

### Oral Formulations

According to the present invention, the route of administration of both COP-1 and IL-4 or IL-10 is preferably oral or enteral. The preferred oral or enteral pharmaceutical formulation may comprise, for example, a pill, a liquid or a capsule containing amounts of COP-1 and IL-4 or IL-10 that are effective in combination to treat Multiple Sclerosis.

Each oral (or enteral) formulation according to the present invention may comprise inert constituents including pharmaceutically acceptable carriers, diluents, fillers, solubilizing or emulsifying agents, and salts, as is well-known in the art. For example, tablets may be formulated in accordance with conventional procedures employing solid carriers well-known in the art. Capsules employed in the present invention may be made from any pharmaceutically acceptable material, such as gelatin, or cellulose derivatives. Sustained release oral delivery systems and/or enteric coatings for orally administered dosage forms are also contemplated, such as those described in U.S. Pat. No. 4,704,295, issued Nov. 3, 1987; U.S. Pat. No. 4,556,552, issued Dec. 3, 1985; U.S. Pat. No. 4,309,404, issued Jan. 5, 1982; and U.S. Pat. No. 4,309,406, issued Jan. 5, 1982.

Examples of solid carriers include starch, sugar, bentonite, silica, and other commonly used carriers. Further non-limiting examples of carriers and diluents which may be used in the formulations of the present invention include saline, syrup, dextrose, and water.

It will be appreciated that the unit content of active ingredient or ingredients contained in an individual dose of each dosage form need not in itself constitute an effective amount, since the necessary effective amount can be reached by administration of a plurality of dosage units (such as capsules or tablets or combinations thereof).

COP-1 and IL-4 or IL-10 may be administered in a single dosage form or in multiple dosage forms. Furthermore, they may be administered separately or together.

COP-1 or Th2-enhancing cytokines can also be administered by inhalation as provided in PCT/US90/07455 (published as WO 91/08760). According to this alternate embodiment of the present invention, administration is in aerosol or inhaled form. The COP-1 or cytokine can be administered as dry powder particles or as an atomized aqueous solution suspended in a carrier gas (e.g., air or N₂).

The pharmaceutical formulations for administration by inhalation of the present invention may include, as optional ingredients, pharmaceutically acceptable carriers, diluents, solubilizing and emulsifying agents, and salts of the type that are well-known in the art. Examples of such substances include normal saline solutions, such as physiologically buffered saline solutions, and water containing between about 1 mg and about 300 mg of the antigens.

Dry aerosol in the form of finely divided solid particles of active substance that are not dissolved or suspended in a liquid are also useful in the practice of the present invention. The active substance may be in the form of dusting powders and comprise finely divided particles having an average particle size of between about 1 and 5 microns, preferably between 2 and 3 microns. Finely divided particles may be prepared by pulverization and screen filtration using techniques well known in the art. The particles may be administered by inhaling a predetermined quantity of the finely divided material, which can be in the form of a powder.

The pharmaceutical formulations of the present invention may be administered in the form of an aerosol spray using for example, a nebulizer such as those described in U.S. Pat. Nos. 4,624,251 issued Nov. 25, 1986; 3,703,173 issued Nov. 21, 1972; 3,561,444 issued Feb. 9, 1971 and 4,635,627 issued Jan. 13, 1971. The aerosol material is inhaled by the subject to be treated.

Specific non-limiting examples of the carriers and/or diluents that are useful in the by-inhalation pharmaceutical formulations include water and physiologically-acceptable buffered saline solutions such as phosphate buffered saline solutions pH 7.0-8.0. Additional non-limiting examples of suitable carriers or diluents for use in by-inhalation pharmaceutical formulations or dosage forms of the present invention are disclosed in U.S. Pat. Nos. 4,659,696, issued Apr. 21, 1987, 4,863,720, issued Sep. 5, 1989 and 4,698,332, issued Oct. 6, 1987.

Other systems of aerosol delivery, such as the pressurized metered dose inhaler (MDI) and the dry powder inhaler as disclosed in Newman, S. P. in Aerosols and the Lung, Clarke, S. W. and Davia, D. eds. pg. 197-224, Butterworths, London, England, 1984, can be used when practicing the present invention.

Aerosol delivery systems of the type disclosed herein are available from numerous commercial sources including Fisons Corporation (Bedford, Mass.), Schering Corp. (Kenilworth, N.J.) and American Pharmoseal Co. (Valencia, Calif.).

Parenteral administration of IL-4 or IL-10 may be via subcutaneous, intramuscular, or intraperitoneal, routes, with subcutaneous being preferred for treatment purposes. In the case of parenteral administration, IL-4 or IL-10 may be formulated in sterile saline or other carriers well known in the art, and may include excipients and stabilizers that are standard in the art.

### Treatment of MS with Combination Therapy

It has been surprisingly discovered that mucosal administration of a COP-1 in conjunction with mucosal or parenteral administration of IL-4 or IL-10, results in a treatment which suppresses the autoimmune reaction in MS and mammalian models therefor. The effect of combination therapy is substantially augmented when compared to the effect of each treatment separately. For example the combination treatment of oral IL-4 or IL-10 with oral COP-1 shows a substantially greater suppressive effect on the clinical score of EAE as compared with COP-1, or cytokine alone.

Suppression of the clinical and histological symptoms of an autoimmune disease occurs after a specific minimum dosage, which, however, varies according to disease, species of mammal, and cytokine. For oral IL-4, the effective dose range for humans in the combination therapy is preferably between 500 and 1,000,000 international units per day, more preferably between about 2,000 and 50,000 international units per day, and most preferably between about 5,000 and about 20,000 international units per day. Similar doses can be employed for IL-10 administration. The maximum dosage is best ascertained by experimentation. It is anticipated that larger doses are permitted but unnecessary.

Parenteral administration of IL-4 may also be used as an adjunct to COP-1 therapy but oral IL-4 is preferred because of the systemic effect of parenteral IL-4. Parenteral IL-4 however, is quite effective in suppressing autoimmune disease. Parenteral dosage for mammals generally can range from about 500 international units of IL-4 to about 1,000,000 international units although the upper limit of this range is best established by experimentation. It is believed that the upper limit is an amount at which the maximum suppressive effect of parenteral IL-4 is observed (i.e., efficacy might not be lost by using higher amounts but they may be unnecessary). Parenteral administration may take place subcutaneously typically once every other day (without limitation) in single or divided doses. Similar dosages and frequencies of administration for IL-10 may be employed.

It is not necessary for the present invention that a dose of IL-4 be effective by itself. Sub-optimal doses of Th-2 enhancing cytokines that would potentiate the effect of COP-1 can be used.

COP-1 is generally administered to treat MS in a dose of 0.01 mg to 1000 mg/day. In one embodiment a dosage in the range of 0.5-50 mg is employed. It is anticipated that lower or higher doses may be permitted and that it is not necessary that the dose of COP-1 be effective by itself.

Establishing the effective dosage range as well as the optimum amount is well within the skill in the art in light of the information given in this section. For example, dosages for mammals, and human dosages in particular are optimized by beginning with a relatively low dose of cytokine and COP-1 (e.g., 1 mg/day of COP-1 and 500 units of IL-4), progressively increasing it (e.g., logarithmically) and measuring a biological reaction to the treatment; for example, (i) measuring induction of regulatory cells (CD4⁺ and/or CD8⁺) (Chen, Y. et al., Science, 255: 1237 (1994)); (ii) measuring reduction in class II surface markers on circulating T-cells; (iii) measuring the number of TGF-β (and/or IL-4 or IL-10) secreting cells; (iv) assessing the number and activation of immune attack T-cells in the blood (e.g., by limiting dilution analysis and ability to proliferate); or, (v) by scoring the disease severity, according to well-known scoring methods (e.g., by measuring the number of attacks, joint swelling, grip strength, stiffness, visual acuity, ability to reduce or discontinue medication). An effective dosage is any dose that causes at least a statistically or clinically significant attenuation in one of these markers and preferably one that attenuates at least one symptom characteristic of MS during the dosing study.

Administration of COP-1 with IL-4 or IL-10 may be carried out once daily for a period of time ranging from 30 days to several months (e.g., 3-6) or even years (e.g., 2-6). If desired, either COP-1 or IL-4 (or IL-10) may be administered singly on some days, and administered in conjunction with the other agent on other days. Therapy may continue indefinitely (unless the obtained benefit does not persist) given the low risk of side effects afforded by the oral route of administration.

Protease inhibitors (such as soybean trypsin inhibitor, aprotinin, antipain) may be added to oral dosage forms containing IL-4 or IL-10 together with COP-1 to increase the absorbed amount. In that case, the dosage of IL-4 may be decreased.

Monitoring of the patient may be desirable in order to optimize the dosage and frequency of administration. The exact amount and frequency of administration to a patient may vary depending on the stage, frequency of manifestation and severity of the patient's disease and the physical condition of the patient, as is well-appreciated in the art. Such optimization is preferably determined on a case-by-case basis. Optimization of the dosage necessary for immune suppression involves no more than routine experimentation, given the guidelines disclosed herein.

Assessment of the disease severity can be accomplished according to well-known methods depending on the type of disease. Such methods include without limitation:

MS: severity and number of attacks over a period of time; progressive accumulation of disability (which can be measured, e.g., on the Expanded Disability Status Scale); number and extent of lesions in the brain (as revealed, e.g., by magnetic resonance imaging); and frequency of autoreactive T-cells.

EAE: limb paralysis which can be scored as follows: 0—no disease; 1—decreased activity, limp tail; 2—mild paralysis, unsteady gait; 3—moderate paraparesis, limbs splayed apart; 4—tetraplegia; and 5—death.

Stabilization of symptoms, under conditions wherein control patients or animals experience a worsening of symptoms, is one indicator of efficacy of a treatment. Another measure of improvement is the ability to reduce or discontinue other medications, e.g., steroids or other anti-inflammatory medications, and biologic response modifiers such as methotrexate, subcutaneous interferon and the like. The optimum dosage of COP-1 and IL-4 or IL-10 will be the one generating the maximum beneficial effect assessed as described above. Clinically significant-attenuation is one observed by a clinician of ordinary skill in the field of MS.

In addition, other cytokine and non-cytokine synergists can be used in the treatment to enhance the effectiveness of mucosally administered COP-1 and administration of a polypeptide having Th2-enhancing cytokine activity. Oral use of other cytokine synergists (Type I interferons) has been described in co-pending U.S. patent application Ser. No. 08/225,372, corresponding to WO 95/27499. Non-limiting examples of non-cytokine synergists for use in the present invention include bacterial lipopolysaccharides from a wide variety of gram negative bacteria such as various subtypes of E. coli and Salmonella (LPS, Sigma Chemical Co., St. Louis, Mo.; Difco, Detroit, Mich.; BIOMOL Res. Labs., Plymouth, Pa.), Lipid A (Sigma Chemical Co., St. Louis, Mo.; ICN Biochemicals, Cleveland, Ohio; Polysciences, Inc., Warrington, Pa.); immunoregulatory lipoproteins, such as peptides covalently linked to tripalmitoyl-S-glycarylcysteinyl-seryl-serine (P₃ C55) which can be obtained as disclosed in Deres et al. (Nature, 342:561 (1989)) or “Braun's” lipoprotein from E. coli which can be obtained as disclosed in Braun Biochim. Biophys. Acta 435:335 (1976). LPS is preferred and Lipid A is particularly preferred because it is less toxic than the entire LPS molecule. LPS for use in the present invention can be extracted from gram-negative bacteria and purified using the method of Galanes et al. (Eur. J. Biochem. 9:245 (1969)) and Skelly et al. (Infect. Immun. 23:287 (1979)). The effective dosage range for non-cytokine synergists for mammals is from about 15 μg to about 15 mg per kg weight and preferably 300 μg-12 mg per kg weight. The effective dosage range for oral Type I interferon for mammals is from 1,000-150,000 units with no maximum effective dosage having been discerned.

### Materials and Methods

In the experiments described below the following materials and methods are used.

Animals. SJL/J mice, 8 weeks of age are obtained from Jackson Laboratories, Bar Harbor, Me. Animals are maintained on standard laboratory chow and water ad libitum. Animals are maintained in accordance with the guidelines for the Committee on Care of Laboratory Animals of the Laboratory Research Council (Pub. #DHEW:NIH, 85-23, revised 1985).

Antigens and Reagents. MBP is purified from brain tissue by the modified method of Deibler et al. (Prep. Biochem. 2:139 (1972)). Protein content and purity are monitored by gel electrophoresis and amino acid analysis. Histone, hen egg lysozyme and ovalbumin are obtained from Sigma (St. Louis, Mo.).

Induction of Tolerance. For oral tolerance or active suppression, mice are fed 0.5 mg of MBP or 0.25 mg COP-1 dissolved in 1 ml phosphate buffered saline (PBS), or PBS alone, by gastric intubation with a 18-gauge stainless steel animal feeding needle (Thomas Scientific, Swedesboro, N.J.). Animals are fed five times at intervals of 2-3 days with the last feeding two days before immunization.

Induction of EAE. For actively induced disease, mice are immunized in the left foot pad with 100 μg of MBP in 0.1 ml of PBS, containing complete Freund's adjuvant (CFA) and 4 mg/ml of Mycobacterium tuberculosis.

Clinical evaluation. Animals are evaluated in a blind fashion every day for evidence of EAE. Clinical severity of EAE is scored as follows: 0, no disease; 1 limp tail; 2, hind limb paralysis; 3, hind limb paraplegia, incontinence; 4, tetraplegia; and 5 death. Duration of disease is measured by counting the total number of days from disease onset (for control mice usually 9 days after active immunization) until complete recovery (or death) for each animal.

Histology. Histologic analysis of pathological changes can be performed in animals with induced EAE. Spinal cords are removed on day 15 after adoptive transfer (or disease induction) and fixed with 10% neutral buffered formalin. Paraffin sections are prepared and stained with Luxol fast blue-hematoxylin and eosin, by standard procedures (Sobel et al. J. Immunol. 132:2393 (1984)). Spinal cord tissue is sampled in an identical manner for each animal and numbers of inflammatory foci per section (clusters of >20 or more aggregated inflammatory cells), in parenchyma and meninges are scored in a blinded fashion (Sobel et al., supra).

Statistical analysis. Clinical scales are analyzed with a two-tailed Wilcoxon rank sum test for score samples, chi square analysis is used in comparing the incidence of disease between groups, and comparison of means is performed by using the Student's t-test. For individual experiments, 5 animals are generally used per group.

The following examples are illustrative of the present invention and do not limit the scope of the invention.

### EXAMPLE 1

### Assay for TGF-β Induction

Measurement of TGF-β Activity in Serum-Free Culture Supernatants

Serum free culture supernatants are collected from tolerized mice as described by Kehri et al. J. Exp. Med. 163: 1037 (1986) or Wahl et al. J. Immunol. 145:2514 (1990). Briefly, modulator cells are first cultured for 8 hours with the antigen (50 μl/ml) in proliferation medium. Thereafter cells are washed three times and resuspended in serum-free medium for the remainder of the 72 hour culture, collected, then frozen until assayed. Determination of TGF-β content and isoform type in supernatant is performed using a mink lung epithelial cell line (American Type Culture Collection, Bethesda, Md. #CCL-64) according to Danielpour et al. (Danielpour et al. J. Cell. Physiol. 138:79 (1989)), and confirmed by a sandwich Enzyme Linked Immunosorbent Assay (ELISA) assay as previously described (Danielpour et al. Growth Factors 2:61 (1989)). The percent active TGF-β is determined by assay without prior acid activation of the samples.

Alternatively, a transwell culture system can be used to indicate the level of TGF-β which is being produced. This culture system measures the production of TGF-β as a function of suppression of cell proliferation.

Such an assay, or similar assays can be used as one means of determining effective immune suppression employing the methods of the invention.

### EXAMPLE 2

### Suppression of EAE in Mice with a Combination of Oral COP-1 and Oral IL-4 or Oral IL-10

The efficacy of combining oral COP-1 with oral IL-4 or IL-10 is shown in the following experiments. The protocol outlined above is followed:

Mouse Groups

Mice are fed five times with

Group 1: ovalbumin (OVA) as a control (500 μg)

Group 2: OVA (1 mg)+IL-4 (1 μg)

Group 3: OVA (1 mg)+IL-10 (1 μg)

Group 4: MBP (500 μg)

Group 5: MBP+IL-4 (1 μg)

Group 6: MBP+IL-10 (1 μg)

Group 7: COP-1 (250 μg)

Group 8: COP-1 (250 μg)+IL-4 (1 μg)

Group 9: COP-1 (250 μg)+IL-10 (1 μg)

Two days after the last feeding, mice are immunized with MBP in CFA. EAE is induced in SJL/J, 8 week old, female mice by immunizing with 100 μg of mouse MBP in 0.1 ml of a suspension containing 4 mg/ml Mycobacterium tuberculosis (MT). This is followed by pertussis toxin injection (150 ng/mouse) on days 0 and 2. Animals are monitored for disease onset for 35 days. Animals are scored for signs of disease every day beginning on day 9 on a scale of 0 to 5.

The results of this experiment show that feeding COP-1+IL-4, or COP-1+ IL-10, significantly delays the onset of disease, decreases fatality, and/or reduces the mean and maximum clinical scores. Furthermore, feeding IL-4 or IL-10 at the foregoing dose, in combination with COP-1, significantly augments the suppressive effect as compared to feeding with COP-1, MBP, or cytokines alone.

### EXAMPLE 3

### Suppression of Multiple Sclerosis by Oral Administration of COP-1 and IL-4

60 patients with the exacerbating-remitting form of MS are randomly divided into three groups. The first group receives, COP-1 orally in doses of 20 mg/day. The COP-1 is administered as described above in phosphate-buffered saline (PBS). The second group receives oral IL-4 in a dosage of 10,000 units per day. The third group receives COP-1 (20 mg) and IL-4 (10,000 units) orally each day in PBS. Each treatment is administered daily for two years.

The clinical status of the patients is evaluated before beginning treatment using the Kurtzke Expanded Disability Status Scale. Patients in each group are evaluated every 3 months during the treatment protocol. Patients taking COP-1 with IL-4 exhibit an improvement in their Kurtzke units scores on the Expanded Disability Status Scale that is substantially greater than that for patients treated with either COP-1 or IL-4 alone.

What is claimed:

1. A method for suppressing autoimmune reaction in a mammal diagnosed with multiple sclerosis said autoimmune reaction being associated with said multiple sclerosis, the method comprising administering to said mammal: (i) via the mucosal route, an amount of COP-1 and (ii) an amount of a non-interferon polypeptide having Th2-enhancing cytokine activity, the amounts of said COP-1 and said polypeptide being effective in combination to reduce said autoimmune response.

2. The method of claim 1 wherein said COP-1 is orally administered.

3. The method of claim 1 wherein the amounts of COP-1 and said polypeptide are substantially more effective in treating said reaction in combination as compared to the treatment effects achieved by administering COP-1 and said polypeptide alone.

4. The method of claim 1 wherein said polypeptide is selected from the group consisting of IL-4 and fragments thereof having Th2-enhancing cytokine activity.

5. The method of claim 4, wherein said polypeptide is IL-4.

6. The method of claim 5, wherein the amino acid sequence of said IL-4 is derived from the same species as said mammal and is orally administered.

7. The method of claim 1 wherein said polypeptide is selected from the group consisting of IL-10 and fragments thereof having Th2-enhancing cytokine activity.

8. The method of claim 7, wherein said polypeptide is IL-10.

9. The method of claim 8, wherein said IL-10 is derived from the same species as said mammal.

10. The method of claim 1 wherein said mammal is a rodent and said disease is a rodent model for multiple sclerosis.

11. The method of claim 1 wherein said mammal is a human and said disease is multiple sclerosis.

12. A mucosally administrable, pharmaceutical composition for the treatment of multiple sclerosis, comprising a combination of COP-1 and IL-4, whereby the amounts of COP-1 and IL-4 are effective in combination for the treatment of multiple sclerosis.

13. The composition of claim 12 comprising an oral pharmaceutical composition.

14. The composition of claim 12, wherein said combination of COP-1 and IL-4 is more effective than either COP-1 or IL-4 alone for the treatment of multiple sclerosis.

15. The composition of claim 12, wherein COP-1 and IL-4 are combined in a tablet.

16. The composition of claim 12, wherein COP-1 and IL-4 are combined in a capsule.

17. An oral, pharmaceutical composition for the treatment of multiple sclerosis, comprising a combination of COP-1 and IL-10, whereby the quantities of COP-1 and IL-10 are effective in combination for the treatment of multiple sclerosis.

18. The composition of claim 17, wherein said combination of COP-1 and IL-10 is more effective than either COP-1 or IL-10 alone for the treatment of multiple sclerosis.

19. The composition of claim 17, wherein COP-1 and IL-10 are combined in a tablet.

20. The composition of claim 17 wherein COP-1 and IL-10 are combined in a capsule.

21. A method for treatment of multiple sclerosis comprising orally administrating an effective amount, in combination, of (1) a mixture of polypeptides consisting essentially of polymers of alanine, glutamic acid, lysine, and tyrosine, in a molar ratio in said mixture of about 6:2:5:1 and (2) IL-4.

22. A method for treatment of multiple sclerosis comprising orally administrating an effective amount, in combination, of (1) a mixture of polypeptides consisting essentially of polymers of alanine, glutamic acid, lysine, and tyrosine, in a molar ratio in said mixture of about 6:2:5:1 and (2) IL-10.
